(19) United States
(12) Patent Application Publication
(10) Pub. No.: US 2008/0003300 A1
(43) Pub. Date: Jan. 3, 2008

(54) Compostions containing Mg/Zn/F-CaP plus inhibitors of pro-inflammatory Cytokines (a combination of a Free-B-Ring flavonoids and a flavan) for osteoporosis prevention, therapy and treatment of bone diseases

(76) Inventor: Maria C. Gaffar, Princeton, NJ (US)

Correspondence Address: ABRAHAM WILSON, 508 ELLIS PARKWAY, PISCATAWAY, NJ, 08854, US

(21) Appl. No.: 11/819,527

(22) Filed: Jun. 28, 2007

Related U.S. Application Data
(60) Provisional application No. 60/817,632, filed on Jun. 30, 2006.

(51) Int. Cl. A61K 31/7048; A61K 31/353; A61K 33/42

(52) U.S. Cl. 424/602; 424/603; 514/456; 514/27

(57) ABSTRACT

A method is described for the treatment of bone disease by oral dosing of a patient with an inhibitor(s) of pro-inflammatory Cytokines admixed in a pharmaceutically acceptable carrier with calcium/phosphate/magnesium/zinc or strontium salt compound ion—a hydroxyapatite matrix.

### RELATED U.S. APPLICATIONS

This application claims priority from U.S. provisional Application Ser. No. 60/817,632 filed Jun. 30, 2006.

### FIELD OF THE INVENTION

This invention relates to the method for the treatment of bone disease wherein a combination of a Free-B-Ring flavonoid and a flavan is admixed in a pharmaceutically acceptable carrier with a calcium/phosphate/magnesium/zinc or strontium salt compound ion, (a hydroxyapatite matrix) in the treatment of the patient.

### THE PRIOR ART

The prior art describes the use of fluoride (in the form of sodium fluoride, NaF or monofluorophosphate, MFP) for osteoporosis therapy. Studies showed that fluoride (F) stimulates bone forming cell in vitro but animal and clinical studies have shown that the effect of F is biphasic: at low concentration it increases bone mass and at high concentration causes bone fractures [Kleerecoper, 1999]. U.S. Pat. Nos. 3,306,824 and 4,265,877 disclosed F in range of 0.25 to 1.0 mg per day. However these preparations cause gastric irritations. A sustained release preparation with calcium has been proposed to reduce irritations (U.S. Pat. Nos. 3,287,219; 4,130,630; 3,345,265; and 4,861,590.

There is a need for non-irritating low dose highly effective preparation for the treatment and prevention of osteoporosis. It has been demonstrated that magnesium (Mg) increase bone mass in humans [Dimai et al 1998] and zinc stimulated bone formation [Yamaguchi 1995].

WOO5032466A (Apr. 14, 2005) by LeGeros describes combined concentrations of Mg, Zn and F ions in a carbonate containing calcium phosphate (CaP) system for osteoporosis prevention, therapy and for bone repair (Mg/Zn/F-CaP). The Mg/Zn/F-CaP preparations have the following advantages: (1) no serious side effects or deleterious effects on bone strength and structure unlike currently FDA approved anti-resorption agents; (2) compositions are similar to bone mineral which is a carbonate apatite containing small amounts of Mg and trace amounts of Zn (Mg and Zn ions are essential components of more than 200 enzyme systems in human); (3) allow slow release of Mg, Zn, F, Ca and P ions; (4) combine ions in concentrations that promote bone formation and minimize bone resorption; (5) allow the incorporation of lower levels of these ions to avoid deleterious side effects found with higher levels; (6) Mg and Zn ions have beneficial effects on collagen and protein formation and balance the F effect on bone apatite formation and crystal size to promote bone formation with higher mineral density and greater bone mass; (7) the three ions have a synergistic effect that allow the rate of bone formation to catch up with the rate of bone resorption, resulting in a net gain of bone mass.

Preliminary results with Mg/Zn/F-CaP in two FDA accepted models for osteoporosis (i.e., mineral-deficiency induced osteoporosis and ovariectomy induced osteoporosis) showed improvement in bone density, bone strength, cortical bone and trabecular bone thickness [LeGeros et al, AADR 2006; LeGeros reports to NIBIB/NIH, 2005].

The recent developments in the understanding of osteoporosis indicate that estrogen deficiency in post-menopausal women is the major cause of osteoporosis and estrogen has anti-inflammatory effects and inhibits the production of pro-inflammatory bone resorptive cytokines such as IL-1, TNF-α, IL-6 [Jika et al 1992]. The functional block of TNF, but not IL-6, prevented bone loss in ovariectomized mice [J Bone Min Res 12:93-5941, 1997]. On the other hand, estrogen deficiency results in the elevation of bone marrow production of pro-osteoclastogenic cytokines such as TNF. Currently available TNF and cytokine activation inhibitors use monoclonal antibodies to block and require parenteral injections with sometimes serious side effects and are not practical for everyday use.

### SUMMARY OF THE INVENTION

Surprisingly, it has been found that combining Mg/Zn/F-CaP preparation with a combination of a flavan and a flavonoid demonstrate a synergistic inhibition on cytokine activation and significantly enhanced the bone density of ovariectomized rats (Sprague-Dawley) compared to those of ovariectomized rats not receiving the combination, above and beyond the effect of Mg/Zn/F-CaP preparation.

### DESCRIPTION OF THE PREFERRED EMBODIMENTS

Structure of Flavans and Flavanoids

The compositions of these flavans/flavanoids are described in US Patent No. 2006/10079467A1. Flavanoids or bioflavonoids are a widely distributed group of natural products, which have been reported to have antibacterial, anti-inflammatory, antiallergic, antimutagenic, antiviral, antineoplastic, anti-thrombic and vasodilatory activity. The structural unit common to this group of compounds includes two benzene rings on either side a 3-carbon ring as illustrated by the following general structural formula:

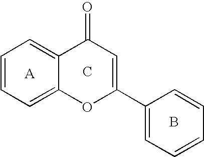

Various combinations of hydroxyl groups, sugars, oxygen and methyl groups attached to this general three ring structure create the various classes of flavonoids, which include flavanols, flavones, flavan-3-ols (catechins), anthocyanins and isoflavones.

Free-Ring flavones and flavonols are a specific class of flavonoids, which have no substituent groups on the aromatic B ring (referred to herein as Free-B-Ring flavonoids), as illustrated by the following general structure: 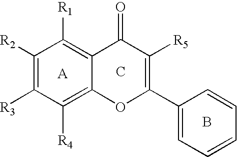
Wherein R₁, R₂, R₃, R₄, R₅ are independently selected from the group consisting of —H, —OH, —SH, OR, —SR, —NH₂, —NHR, —NR₂, —NR₃⁺X⁻, a glycoside of a single or combination of multiple sugars, wherein said glycoside is linked to the 7-hydroxy chromone by a carbon, oxygen, nitrogen or sulfur, and wherein said single or combination of multiple sugars include, but are not limited to aldopentoses, methyl-aldopentoses, aldohexoses, ketohexose and their chemical derivatives thereof;

Wherein

R is an alkyl group between 1-10 carbon atoms, and

X is selected from the group of pharmaceutically acceptable counter anions including, but not limited to hydroxyl, chloride, iodide, fluoride, sulfate, phosphate, acetate, carbonate, etc.

Free-B-Ring flavonoids are relatively rare. Out of 9,396 flavonoids synthesized or isolated from natural sources, only 231 Free-B-Ring flavonoids are known (The Combined Chemical Dictionary, Chapman & Hall/CRC, Version 5:1 June 2001). Free-B-Ring flavonoids have been reported to have diverse biological activity. Typically, flavonoids have been tested for biological activity randomly based upon their availability. Occasionally, the requirement of substitution on the B-ring has been emphasized for specific biological activity, such as the B-ring substitution required for high affinity binding to p-glycoprotein (Boumendjel et al (2001) Bioorg, Med. Chem. Lett. 11 (1):75-77; cardiotonic effect (Itoigawa et al (1999) J. Ethnopharmacol. 65 (3):267-272), protective effect on endothelial cells against linoleic acid hydroperoxide-induced toxicity (Kaneko and Baba (1999) Biosci Biotechnol. Biochem 63 (2):323-328), COX-1 inhibitory activity (Wang (2000) Phytomedicine 7:15-19) and prostaglandin endoperoxide synthase (Kalkbrenner et al (1992) Pharmacology 44 (1): 1-12). Few publications have mentioned the significance of the unsubstituted B ring of the Free-B-Ring flavonoids. One example is the use of 2-phenyl flavones, which inhibit NADPH quinone acceptor oxidoreductase, as potential anti-coagulants. (Chen et al (2001) Biochem. Pharmacol. 61 (11):1417-1427).

The mechanism of action of the anti-inflammatory activity of various Free-B-Ring flavonoids has been controversial. The anti-inflammatory activity of the Free-B-Ring flavonoids, chrysin (Liang et al. (2001) FEBS Lett. 496(1):12-18, wogonin (Chi et al (2001) Biochem. Pharmacol. 61:1195-1203) and halangin (Raso et al (2001) Life Sci. 68 (8):921-931), has been associated with the suppression of inducible cyclooxygenase and nitric oxide synthase via activation of peroxisome proliferator activated receptor gamma (PPARγ) and influence on degranulation and AA release (Tordera et al (1994) Z. Naturforsch [C] 49:235-240). It has been reported that a oroxylin, baicalein and wogonin inhibit the activity of 12-lipoxygenase without affecting cyclooxygenase (You et al. (1999) Arch. Pharm. Res. 22(1):18-24). More recently, the anti-inflammatory activity of wogonin, baicalin and baicalein has been reported as occurring via inhibition of inducible and cox2-gene expression induced by nitric oxide inhibitors and lipopolysaccharide (Chen et al. (2001) Biochem. Pharmacol. 61(11):1417-1427). It has also been reported oroxylin acts via suppression of NFkB activation (Chen et al. (2001) Biochem. Pharmacol. 61(11): 1417-142). Finally, wogonin reportedly inhibits inducible PGE2 production in macrophages (Wakabayashi and Yasui (2000) Eur. J. Pharmacol. 406(3):477-481).

The Chinese medicinal plant, Scutellaria baicalensis contains significant amounts of Free-B-Ring flavonoids, including baicalein, baicalin, wogonin and baicalenoside. Traditionally, this plant has been used to treat a number of conditions including clearing away heat, purging fire, dampness-warm and summer fever syndromes; polydipsia resulting from high fever, carbuncle, sores and other pyrogenic skin infections; upper respiratory infections, such as acute tonsillitis, laryngopharyngitis and scarlet fever; viral hepatitis; nephrities, pelvitis; dysentery; hematemesis and epistaxis. This plant has also been traditionally been used to prevent miscarriage. (Encyclopedia of Chinese Traditional Medicine, Shanghai Science and Technology Press, Shanghai, China, 1998). Clinically, Scutellaria, has a new use to treat conditions such as pediatric pneumonia, pediatric bacterial diarrhea, viral hepatitis, acute gallbladder inflammation, hypertension, topical acute inflammation, resulting from cuts and surgery, bronchial asthma and upper respiratory infections. (Encyclopedia of Chinese Traditional Medicine, Shanghai Science and Technology Press, Shanghai, China, 1998). The pharmacological efficacy of Scuttellaria roots for treating bronchial asthma is reportedly related to the presence of Free-B-Ring flavonoids and their suppression of eotaxin associated recruitment of eosinophils. (Nakajima et al. (20001) Planta Med. 67(2):132-135).

To date, a number of naturally occurring Free-B-Ring flavonoids have been commercialized for varying uses. For example, liposome formulations of Scutellaria extracts have been utilized for skin care (U.S. Pat. Nos. 5,643,598; 5,443,983). Baicalin has been used for preventing cancer due to its inhibitory effects on oncogenes (U.S. Pat. No. 6,290,995). Baicalin and other compounds have been used as antiviral, antibacterial and immunomodulating agents (U.S. Pat. No. 6,083,921 and WO98/42363) and as natural anti-oxidants (WO98/49256 and Poland Pub. No. 9,849,256). Flavonoids formulates with terpoids have been used for treating and inhibiting dental carries (US#20040057908). Japanese Pat. No. 63027435 describes the extraction, and enrichment of baicalein and Japanese Pat. No. 61050921) describes the purification of baicalin.

U.S. application Ser. No. 10/091,362, filed Mar. 1, 2002, entitled “Identification of Free-B-Ring Flavonoids as Potent COX-2-inhibitors,” and U.S. application Ser. No. 10/427,746, filed Jul. 22, 2003, entitled “Formulation of a Mixture of Free-B-Ring Flavonoids and Flavans as a Therapeutic Agent” disclose a method for inhibiting the cyclooxygenase enzyme COX-2 by administering a composition comprising a Free-B-Ring flavonoids or a composition containing a mixture of Free-B-Ring flavonoids to a host in need thereof. This is the first report of a link between Free-B-Ring flavonoids and COX-2 inhibitory activity. These applications are specifically incorporated herein by reference in their entirety.

Flavans include compounds illustrated by the following general structure:

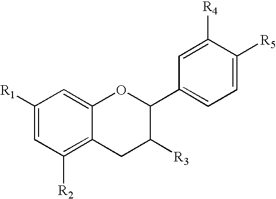

Wherein

R₁, R₂, R₃, R₄, and R₅ are independently selected from the group consisting of H, —OH, —SH, OCH₃, —SCH₃, —OR, —SR— NH₂, —NRH, —NR₂, —NR₃, ⁺X⁻, esters of the mentioned substitution groups including but not limited to gallate, acetate, cinnamoyl and hydroxyl-cinnamoyl esters, trihydroxybenzoyl esters and caffeoyl esters, and their chemical derivatives thereof, a glycoside of a single or combination of multiple sugars, therein said glycoside is linked to the 7-hydroxy chromone by a carbon, oxygen, nitrogen or sulfur, and wherein said single or combination of multiple sugars include, but are not limited to aldopentoses, methyl-aldopentose, aldohexoses, ketohexose and their chemical derivatives thereof and other polymerized flavan,

wherein,

R is an alkyl group having between 1-10 carbon atoms, and

X is selected from the group of pharmacologically acceptable counter anions including but not limited to hydroxyl, chloride, iodide, sulfate, phosphate, acetate, fluoride, and carbonate, etc.

Catechin is a flavan, found primarily in green tea, having the following structure:

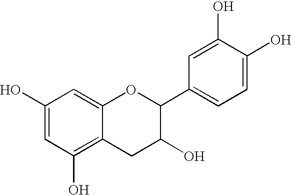

Catechin works both alone and in conjunction with other flavonoids found in tea, and has both antiviral and antioxidant activity. Catechin has been shown to be effective in the treatment of viral hepatitis. It also appears to prevent oxidative damage to the heart, kidney, lungs and spleen and has been shown to inhibit the growth of stomach cancer cells.

Catechin and its isomer epicatechin inhibit prostaglandin endoperoxide synthase with an IC₅₀ value of 40 μM. (Kalkbrenner et al. (1992) Pharmacol. 44:1-12). Commercially available pure (+)-catechin inhibits COX-1 with an IC₅₀ value of around 183 to 279 μM depending upon the experimental conditions, with no selectivity for COX-2. (Noreen et al. (1998) J. Nat. Prod. 61:1-7). Green tea catechin, when supplemented into the diets of Sprague Dawley male rats, lowered the activity level of platelet PLA₂ and significantly reduced platelet cyclooxygenase levels. (Yan et al. (1999) J. Nutr. Sci. Vitaminol. 45:337-346). Catechin and epicatechin reportedly weakly suppress COX-2 gene transcription in human colon cancer DLD-1 cells (IC₅₀ 8=415.3 μM). (Mutoh et al. (2000) Jpn. J. Cancer Res. 91:686-691). The neuroprotective ability of (+)-catechin from red wine results from the antioxidant properties of catechin, rather than inhibitory effects on intracellular enzymes, such as cyclooxygenase, lipoxygenase, or nitric oxide synthase (Bastianetto et al. (2000) Br. J. Pharmacol. 131:711-720). Catechin derivatives purified from green and black tea, such as epigallocatechin-3-gallate (EGCG), epigallocatechin (EGC), the flavins showed inhibition of cyclooxygenase and lipoxygenase dependent metabolism of AA in human colon mucosa and colon tumor tissues (Hong et al. (2001) Biochem. Pharmacol. 62:1175-1183) and induce COX-2 expression and PGE₂ production (Park et al. (2001) Biochem. Biophys. Res. Commun. 286:721-725).

Acacia is a genus of leguminous trees and shrubs. The genus Acacia includes more than 1000 species belonging to the family of Leguminosae and the subfamily of Mimosoideae. Acacias are distributed widely in tropical and subtropical areas of Central and South America, Africa, parts of Asia, as well as Australia, which have the largest number of endemic species. To date, approximately 330 compounds have been isolated from various Acacia species. Flavonoids are the major class of compounds isolated from Acacias. Approximately 180 different flavonoids have been identified, 111 of which are flavans. Terpenoids are second largest class of compounds isolated from species of the Acacia genus with 48 compounds having been identified. Other classes of compounds isolated from Acacia include alkaloids (28), amino acids/peptides (20), tannins (16), carbohydrates (15), oxygen heterocycles (15) and aliphatic compounds (10). (Buckingham, The Combined Chemical Dictionary, Chapman & Hall CRC, version 5:2, December, 2001).

The Uncaria genus, includes 34 species many of which are well known as medicinal plants. Uncaria plants have been utilized by different cultures for treatment of wounds, and ulcers, fevers, headaches, gastrointestinal illnesses and microbial/gungal infections. Uncaria plants contain significant amounts of catechin and other flavones. Other components that have been reported in Uncaria genus include alkaloids, terpenes, quinovic acid glycosides, coumarins and flavonoids. Uncaria gambir is a species common in Malaysia, Singapore, India and other South East Asian countries. Catechins are major components in the whole plant of Uncaria gambir.

### EXAMPLES OF THE INVENTION

### Example I

Mg/Zn/F-CaP with Flavans and Flavonoids

For this study, the composition (in wt %) of the Mg/Zn/F-CaP (#86) was: calcium (Ca)=23.74, phosphorous (P)=13.38; magnesium (Mg)=2.32; zinc (Zn)=2.18; fluoride (F)=0.90. X-Ray diffraction profile (FIG. 1) and FT-IR spectrum (FIG. 2) show that the preparation is a carbonate apatite similar to bone mineral [LeGeros 1981].

The addition of these flavans and flavonoids unexpectedly improved osteoporosis lesions in vivo above and beyond Zn/Mg/F-CaP. The utility of the invention is illustrated from the following in vivo experiments in ovarietomized rats.

Ovariectomized (OVX) rats obtained from Charles River were divided into 3 groups: (1) OVX group receiving the normal diet; (2) Blue group: OVX rats receiving the combination supplement (#85+FV); and (3) Yellow group: OVX rats receiving only Mg/Zn/F-CaP (#86). The amount of Mg/Zn/F-CaP (#86) mixed in the Yellow Group diet was 1.2 wt % of the normal diet. The amount of flavan/flavonoid (FV) mixed with #86 and added to the Blue Group diet: 0.016 wt % of the total diet. The average daily consumption of each rat was 30g of the diet and ad libitum water

Analyses using radiography (Faxitron), computer tomography (Micro-CT) and scanning electron microscopy (SEM) showed significant improvement in bone properties (bone density, cortical bone and trabecular bone thickness, and bone strength) of OVX rats receiving the combination (Mg/Zn/F-CaP+flavan/flavonoids) supplement compared to OVX rats not receiving the supplement. A synergistic effect of Mg/Zn/F-CaP and FV on bone properties was observed on rats receiving the combined supplement compared to those receiving the Mg/Zn/F-CaP supplement alone (FIGS. 3, 4 and Table 1).

TABLE 1
Micro-CT measurements

| Sample | TV | BV | BV/TV | CD | TbTh | TbSp | TbN |
|---|---|---|---|---|---|---|---|
| A | 20.12 | 7.62 | 0.379 | 93.5 | 0.10 | 0.18 | 4.80 |
| B | 27.96 | 2.03 | 0.074 | 14.9 | 0.08 | 1.02 | 1.63 |
| C | 28.76 | 5.59 | 0.19 | 46.54 | 0.09 | 0.73 | 1.43 |
| D | 29.29 | 3.99 | 0.14 | 29.89 | 0.09 | 0.93 | 1.25 |

(A = non-OVX; B = OVX; C = Mg/Zn/F-CaP + FV;
D = Mg/zn/F-CaP
TV = total volume;
BV = bone volume;
CD = connectivity density;
TbTh = trabecular thickness;
TbSp = trabecular space;
TbN = trabecular number)

### Example II

The Effect of Mixed Flavans/Flavanoids on TNFα Release from Stimulated Peripheral Blood Monocytes (PBMCS) as Measured by TNFα Gene Expression

The cells were cultured in RPMI 1640 supplemented with 1% serum albumin for approximately 12 hours before treating with LPS (Lipopolysaccharide from E. Coli) at increasing concentrations to induce TNFα release. A wide range of LPS from 3 to 100 micrograms (μg) per ml indicated that this mixture is highly effective in releasing a key cytokine involved in bone loss. The release of cytokine is measured by gene expression which indicates the release of TNFα. Concentration of 10 μg/per ml showed the highest release of TNFα. A mixture of flavans and flavonoids inhibited the gene expression induced by LPS as shown in the Table below. TNFα gene expression is normalized to 100% without LPS. When LPS is present the expression increases 10-fold to 1000%. With 3 to 30 μg per ml of flavan/flavonoids+LPS, TNFα gene expression is restored to baseline.

TNFα Gene Expression

| | Concentration | |
|---|---|---|
| Agent | (ug/ml) | TNFα Gene Expression |
| LPS | 0 | 100% |
| LPS | 3 | 1000% |
| LPS + Flavan/Flavonoid | 3 | 100% |
| (3 μg/ml) | | |
| LPS + Flavan/Flavonoid3 | | 100% |
| (30 μg/ml) | | |

### Example III

### Examples of Preparations Containing Mg/Zn/F-CaP with Flavans/Flavonoids

The concentrations of Mg, Zn and/or F in the carbonate-containing calcium phosphate matrix may be increased or decreased compared to the concentrations in preparation #86 used in the preliminary study.

The concentration of the Mg/Zn/F-CaP supplement in the diet can be lower or higher than the one used in the preliminary study.

The concentration of the flavan/flavonoids added to the Mg/Zn/F-CaP can be lower or higher than the one used in the study.

Examples of regular tablets and sustained release tablets are described below. No sintering or heating is required for regular tablets. The Mg/Zn/F-CaP plus flavans/flavonoids are from 0.001 to 10.0%.

The means for providing controlled (i.e., sustained) release of the active ingredient may be selected from any of the known sustained-release oral drug delivery system. Of the known sustained-release delivery systems, the preferred system is the Forest Synchtron drug delivery system in which the active ingredient is dispersed uniformly and homogenously throughout a matrix of water swellable modified cellulosic powder or fibers forming coherent network, as a matrix. Soft gelatin capsules are prepared as follows:

| | |
|---|---|
| Mg/Zn/F-CaP plus flavan/flavonoid* | 0.0001 to 10% |
| Citric acid anhydrous | 0.015 mg |
| Propyl gallate | 0.020 mg |
| Vitamin E (dl-alpha tocopherol) | 0.020 mg |
| Alcohol, dehydrated | 1.145 mg |
| Vegetable oil | 98.800 mg |
| Total fill weight per capsule: | 100.0 mg |

*Includes 10% excess

Compressed Tablet

Mg/Zn/F-CaP//flavan/flavonoids combinations are adsorbed onto a suitable substrate (cellulosic material) and progressively diluted (triturated) and dispersed in a calcium carbonate (CaCO3) granules containing the equivalent of 250 mg to 500 mg of calcium. The granules are compressed into a tablet.

Film-Coated Tablet

Mg/Zn/F-CaP//flavan/flavonoid combinations are dispersed or dissolved in an organic solvent or aqueous mix, admixed with a pharmaceutically acceptable polymer and spray coated onto a table core containing CaCO3 (in an amount equivalent to 250 mg to 500 mg of calcium). Additional protective coating may also be added.

Dry-Filled Capsule

(a) Mg/Zn/F-CaP//flavan/flavonoids combinations are adsorbed onto a suitable substrate and placed in a capsule. The remainder of the capsule is filled with a suitable form of CaCO3 (containing the equivalent of 250 mg to 500 mg of calcium).
(b) Microencapsulated amounts of 5-40 ug (mcg) of Mg/Zn/F-CaP//flavan/flavonoids combinations are placed in a capsule and the remainder of the capsule is filled with a suitable form of CaCO3 (containing the equivalent of 250 mg to 500 mg of calcium).
(c) A pellet or tablet containing 5-40 ug (mcg) of Mg/Zn/F-CaP/flavan/flavonoids combinations are placed in a capsule and the remainder of the capsule is filled with a suitable form of CaCO3 (containing the equivalent of 250 mg to 500 mg of calcium).
   Soft Gelatin Capsules

A non-aqueous slurry containing 5-40 ug (mcg) of Mg/Zn/F-CaP//flavan/flavonoids combination is filled into a soft gelatin capsule.

Liquid-Filled Hard Capsules

A non-aqueous slurry containing 5-40 ug (mcg) of Mg/Zn/F-CaP/flavan/flavonoids combination is filled into a hard gelatin capsule.

Granules/Powders

(a) A suitably flavored granulate containing 5-40 ug (mcg) of Mg/Zn/F-CaP//flavan/flavonoids combination is filled into a sachet. This formulation is suitable for dispersing in a glass of beverage (e.g., water, milk or fruit juice) for immediate consumption.
(b) A unit dose cup containing containing 5-40 ug (mcg) of Mg/Zn/F-CaP/flavan/flavonoids combination and CaCO3 (containing the equivalent of 250 mg to 500 mg of calcium) in granulate/powder form comprising also flavors, thickeners and possibly other excipients. The product is suitable for reconstitution with water to be shaken and drunk as a suspension.

### BRIEF DESCRIPTION OF THE FIGURES

FIG. 1: X-ray diffraction profile of Mg/Zn/F-CaP preparation #86 showing an apatite diffraction pattern similar to that of bone apatite.

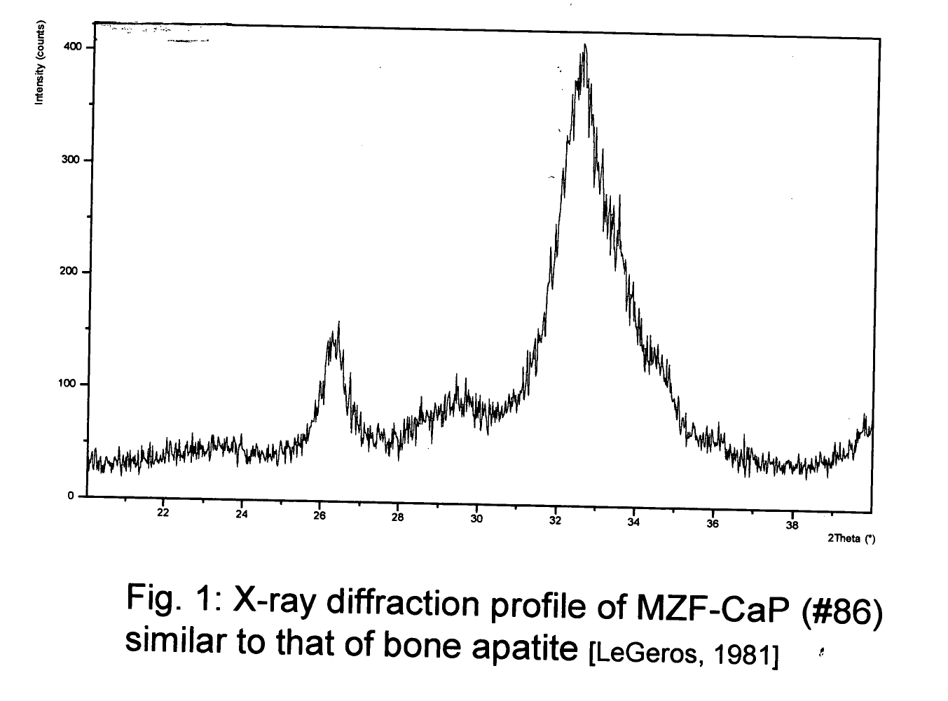

FIG. 2: FT-IR spectrum of Mg/Zn/F-CaP preparation #86 showing characteristic spectrum of carbonate apatite [LeGeros et al 1971]

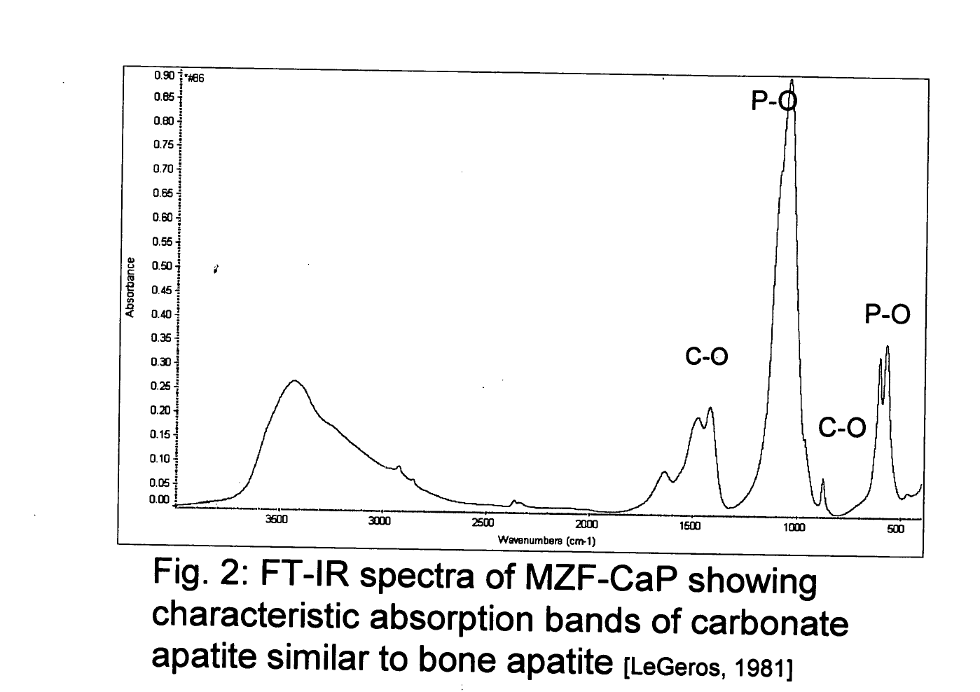

FIG. 3: Micro CT of 200 slices from femur head (A) OVX on basic diet; (6) OVX on (basic diet+MZF-CaP (#86) with flavan/flavonoids); (C) OVX on (basic diet+MZF-CaP (LeGeros et al 2006)

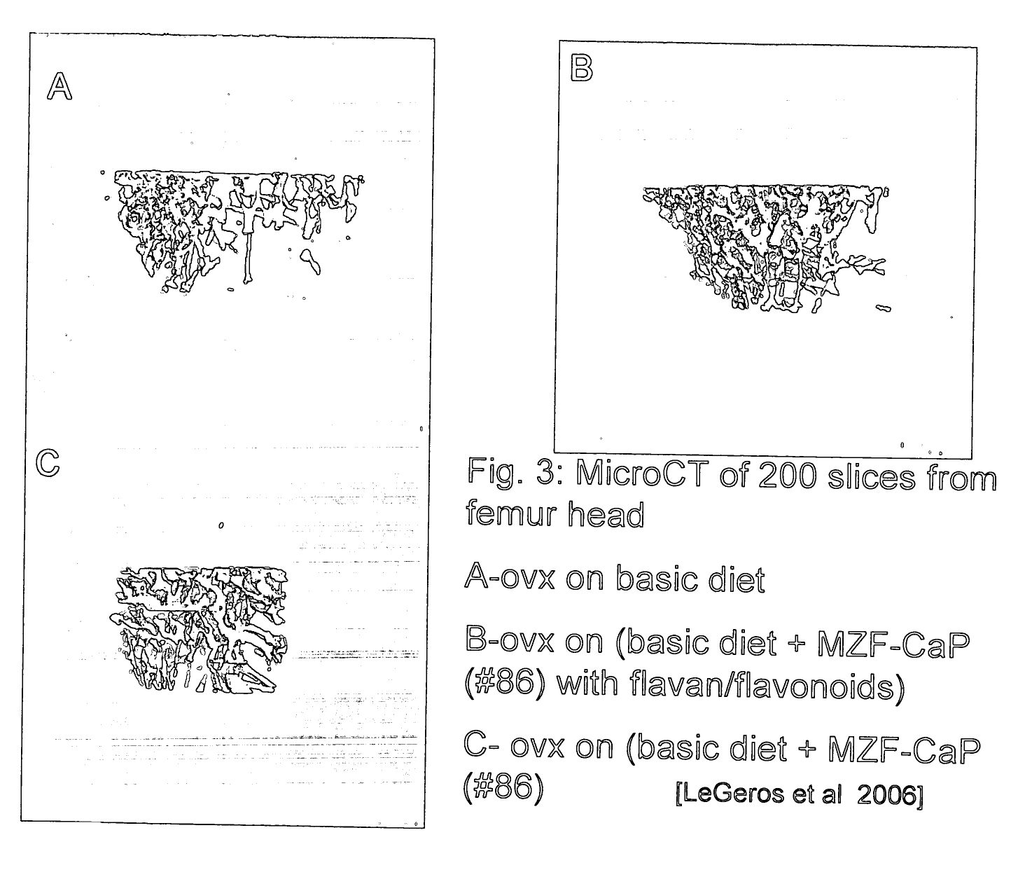

FIG. 4: Micro-CT of femur head (A) OVX on basic diet; (6) OVX on (basic diet+MZF-CaP (#86) with flavan/flavonoids (C) OVX on basic diet+MZF-CaP (#86) (LeGeros et al 2006)

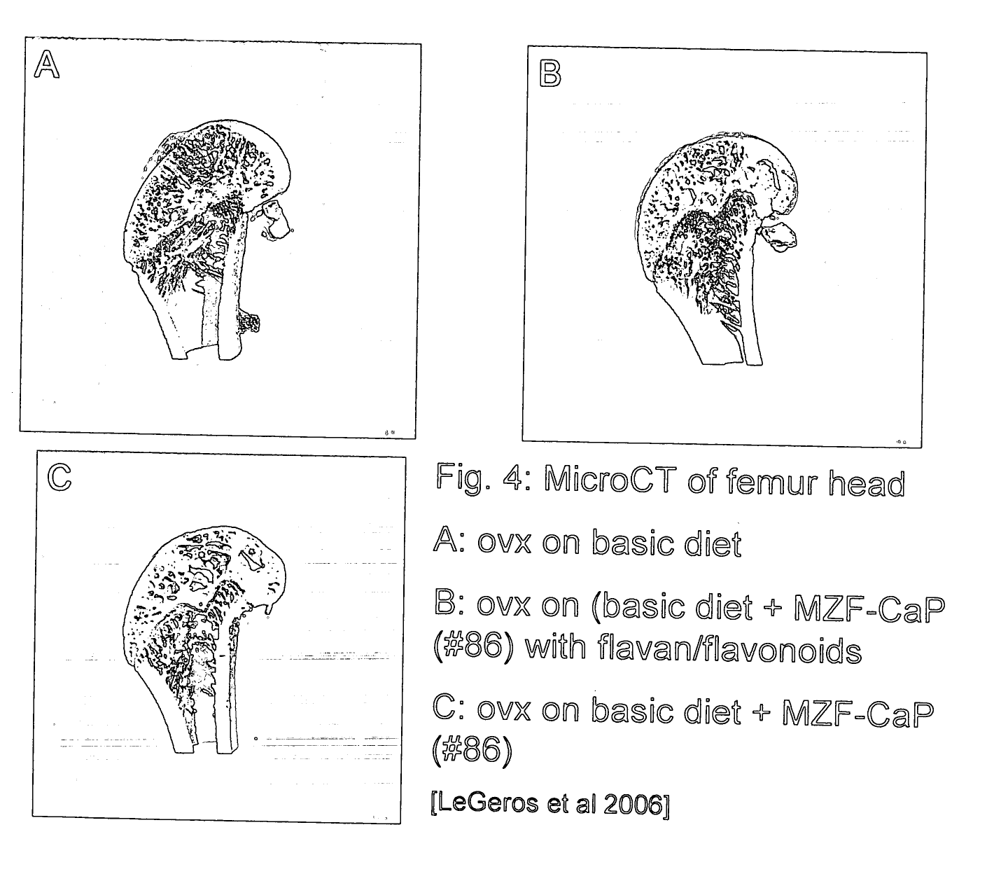

What is claimed is:

1. A method for the treatment of bone disease and prevention of bone diseases, including bone loss from osteoporosis, bone repair of fractures and osteomalacia, which comprises a Free B-Ring flavonoid and at least a flavan in an admixture with a calcium/phosphate system containing one or more ions selected from the group consisting of magnesium, zinc and fluoride ions forming a matrix for patient ingestion.

2. The method of claim 1 wherein the ratio of Free-B-Ring flavonoid to flavan in said compositions is selected from the range of 99:1 Free-B-Ring flavanoid:flavan to 1:99 of Free-B-Ring flavanoid:flavan.

3. The method of claim 2 wherein the ratio of Free-B-Ring flavanoid:flavan in the composition of matter is about 80:20.

4. The method of claim 1 wherein said Free-B-Ring flavonoid is a compound having the following structure: 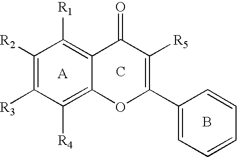
Wherein
R₁, R₂, R₃, R₄ and R₅ are independently selected from the group consisting of —H, —OH, —SH, —OR, —NH₂, —NHR, —NR₂, —NR₃ ^{+X}⁻, a glycoside of a single or combination of multiple sugars, wherein said glycoside is linked to the 7-hydroxy chromone by a carbon, oxygen, nitrogen or sulfur, and wherein said single or combination of multiple sugars include, but are not limited to aldopentoses, methyl-aldopentose, aldohexoses, ketohexose and their chemical derivatives thereof,
wherein
R is an alkyl group having between 1-10 carbon atoms; and
X is selected from the group of pharmaceutically acceptable counter anions including, hydroxyl, chloride, iodide, sulfate, phosphate, acetate, fluoride, carbonate and strontium.

5. The method of claim 1 wherein said flavan is selected from the group of compounds having the following structure: 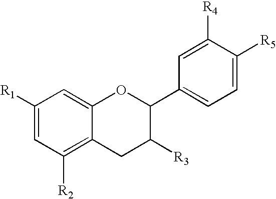
wherein
R₁, R₂, R₃, R₄ and R₅ are independently selected from the group consisting of —H, —OH, —SH, —OCH₃, —SCH₃, —OR, —NH₂, —NRH, —NR₂, —NR₃⁺X⁻, esters of the mentioned substitution groups, including but not limited to, gallate, acetate, cinnamoyl and hydroxylcinnamoyl esters, and their chemical derivatives thereof; a glycoside of a single or combination of multiple sugars, wherein said glycoside is linked to the 7-hydroxy chromone by a carbon, oxygen, nitrogen or sulfur, and wherein said single or combination of multiple sugars include but are not limited to aldopentoses, methyl-aldopentose, aldohexoses, ketohexose and their chemical derivatives thereof and other polymerized flavans;
wherein
R is an alkyl group having between 1-10 carbon atoms; and X is selected from the group of pharmaceutically acceptable counter anions including but not limited to hydroxyl, chloride, iodide, sulfate, phosphate, acetate, fluoride, carbonate.

6. The method of claim 1 wherein the Free-B-Ring flavonoid and the flavan are isolated from a plant.

7. The method of claim 6 wherein the Free-B-Ring flavonoid and the flavan are isolated from a plant part selected from the group consisting of stems, stem barks, trunks, trunk barks, twigs, tubers, roots, root barks, young shoots, seeds, rhizomes, flowers and other reproductive organs, leaves and other aerial parts.

8. The method of claim 6 wherein said Free-B-Ring flavonoid is isolated from a plant family selected from the group consisting of Annonaceae, Asteraceae, Bignoniaceae, Combretaceae, Compositae, Euphorbiaceae, Labiatae, Lauranceae, Leguminosae, Moraceae, Piniaceae, Pteridaceae, Sinopteridaceae, Ulmaceae and Zingiberaceae.

9. The method of claim 6 wherein the Free-B-Ring flavonoid is isolated from a plant genus selected from the group consisting of Desmos, Achyroclile, Oroxylum, Buchenavia, Anaphalis, Cotula, Gnaphalium, Helichrysum, Centaurea, Eupatorium, Baccharis, Sapium, Scutellaria, Molsa, Colebrookea, Stachys, Origanum, Ziziphora, Lindera, Actinodaphne, Acacia, Derris, Glycyrrhiza, Milleftia, Pongamia, Tephrosia, Artocarpus, Ficus, Pityrogramma, Notholaena, Pinus, Ulmus and Alpina.

10. The method of claim 6 wherein the flavan are isolated from a plant genus selected from the group consisting of the Acacia and Uncaria.

11. The method of claim 6 wherein the Free-B-Ring flavonoid is isolated from a plant or plants in the Scutellaria baicalensis and Oroxylum indicum and said flavan is isolated from a plant or plants in the Acacia catechu and Uncaria gambir.

12. The method of claim 1 wherein calcium/phosphate/zinc/magnesium is a carbonate apatite

13. The method of claim 1 wherein the composition is present in preparations administered to a host in need thereof at a concentration of 0.0001% to about 10% of the preparation.

14. The method of claim 1 wherein the matrix is a tablet.

15. The method of claim 1 wherein the matrix is a sustained release oral drug delivery system.
